(19) United States
(12) Patent Application Publication
(10) Pub. No.: US 2012/0316522 A1
(43) Pub. Date: Dec. 13, 2012

(54) EXTERNAL CATHETER

(75) Inventors: Ryan J. Carter, St. Louis, MO (US); Kyle D. Miller, Charlotte, NC (US); Aaron Brookhart, St. Louis, MO (US)

(73) Assignee: MLP PRODUCTS, St. Louis, MO (US)

(21) Appl. No.: 13/491,714

(22) Filed: Jun. 8, 2012

Related U.S. Application Data
(60) Provisional application No. 61/494,508, filed on Jun. 8, 2011.

(51) Int. Cl. A61F 5/453; A61F 5/457

(52) U.S. Cl. 604/353; 604/349

(57) ABSTRACT

An external catheter includes a groin piece that is anatomically shaped to a user's pelvis and rests on the user's pelvis. A flange extends from the groin piece and defines an opening through the groin piece. The opening is sized to receive the user's penis therethrough. At least one indentation is formed at least partially around the flange. A removable tube is attached to the flange. The tube is sized to receive the penis extending through the opening. A locking ring is positioned in the at least one indentation to secure the tube to the flange.

### CROSS-REFERENCE TO RELATED APPLICATIONS

This application claims priority to U.S. Provisional Patent Application Ser. No. 61/494508 filed Jun. 8, 2011 and titled “PORTABLE URINATION DEVICE”, which is incorporated in its entirety herein.

### STATEMENT REGARDING FEDERALLY SPONSORED RESEARCH

Not Applicable.

### APPENDIX

Not Applicable.

### BACKGROUND OF THE INVENTION

1. Field of the Invention

The present invention relates to an external catheter, and more particularly to an external catheter that has a tube extending down the leg of the user.

2. Related Art

Portable urination devices have been used for people who cannot get to a restroom, who do not want to touch the facilities at a filthy public restroom, or who are otherwise in a situation where removing one's clothes is difficult. Most portable urination devices either have a container that captures the urine for later disposal or an opening proximate to the user's groin region that directs the urine away from the user's body for immediate disposal in a facility or on the ground. Generally, known portable urination devices are bulky and easily noticeable when worn. For example, several known portable urination devices include a funnel that extends outward from the user's pelvis. Additionally, known portable urination devices are unsanitary. These devices typically include a funnel or cup-like apparatus that the user urinates into. The urine may then be directed out of the cup. However, not all of the urine drains from the cup and the urine is left in the user's pants. This urine may leak out of the cup and into the user's pants or may cause bacteria to grow within the cup. Accordingly, known portable urination devices require frequent cleaning to rid the device of bacteria.

There is a need for an external catheter that has a low profile that prevents the catheter from being noticed when in use. There is also a need for an external catheter that prevents urine from collecting within the user's pants. Further, a need exists for an external catheter that includes disposable parts so that the catheter does not require frequent cleaning to prevent the spread of bacteria.

### SUMMARY OF THE INVENTION

The present invention is an external catheter that allows hunters, skiers, or anyone who is away from sanitary restroom facilities to relieve themselves at their discretion, having the urine flow through a tube running along the entire length of the user's leg toward the user's ankle and foot. The tube runs out near the user's shoe, and a targeted lift of the leg allows for clean dismissal. When in the wild, the user may stand with both feet on the ground and let the urine flow out of the end of the tube directly on the ground or may elevate their foot on a log, rock or other surface to direct the urine away from the user's shoe to the ground. Similarly, when the facilities in a restroom are filthy, the user can elevate their foot and place it on the lip of a toilet, urinal or other fixture to direct the urine into the bowl, trough or other disposal element.

In the preferred embodiment, the distal end of the tube is strapped or otherwise secured to the ankle, lower leg or foot of the user which keeps the tube from riding up the pant leg as the person sits, walks or otherwise changes positions. According to the present invention, the person can relieve themselves while being fully dressed, without undressing or touching any fixture and without needing to carry the expelled urine in a container.

The external catheter of the present invention has two primary parts, an anatomically-shaped groin piece and a tube connected to the groin piece. The groin piece is attached to a strap or is otherwise incorporated into or fixed to a wearable undergarment. The groin piece is preferably made from flexible but rigidly shaped silicone which is anatomically shaped to rest on the user's pelvis and has an opening through which the user's penis is extended. The tube is removeably attached to the groin piece through a leak-proof fitting. The length of the tube may be variable for differently sized users, but in the preferred embodiment, the user will select the particular length that fits their body. The external catheter may be created with a tube length that would be long enough to fit tall users, such as a 42 inch length, and shorter users may cut the end of the tube to fit their particular size. Accordingly, the elongated tube extends from a top end proximate to the groin piece down past the calf of the user to a bottom end proximate to the ankle of the user.

As described above, the tube drains directly on the ground. The tube is preferably sturdy enough to avoid damage that could cause leaks and flexible enough so that it fits comfortably beneath a user's outer garments. The groin piece preferably has a securing mechanism in the form of a strap that secures the groin piece to the user. It will be appreciated that the strap may be attached to the user's belt or a button of the user's pants. In one embodiment, the groin piece may be integrally formed within an undergarment, for example, underwear or a jock strap.

It will be appreciated that the present invention could have a number of uses that are not necessarily condoned by societal norms, but such uses are within the scope of the claims to the present invention. For example, some people may have a fetish or other obsession with urinating in semi-public areas but who want to be discreet, such as persons who urinate in their garden or yard. Also, revelers and other party-goers may want to relieve themselves while in the fresh air at an outdoor festival without needing to go inside a cramped, portable toilet facility that is usually filthy and stench-ridden.

With the introduction and adoption of the present invention as a reliable way to efficiently and safely urinate at public events, it is also anticipated that a number of venues could encourage the use of the device by offering urination grates or holes within their restroom facilities. The user could stand over the grate or to one side of the hole as their urine drains out the end of the tube directly into the facility. The people do not need to undress or touch anything in the restroom with their hands, speeding the process of urination and making it a more sanitary environment.

Alternatively, the present invention may be used as a medical device for those who are incontinent. For example, since it is already known to attach a container or other collection device to a urination device at the end of a tube, it will be appreciated that the present invention could also be used with a soft-sided container or other conformable collection device that could be strapped to a user's leg below the knee or calf. The collection device may be emptied into a toilet using a valve positioned at a bottom of the collection device.

### BRIEF DESCRIPTION OF THE DRAWINGS

The present invention will become more fully understood from the detailed description and the accompanying drawings, wherein:

FIG. 1 is a front perspective view of three versions of a groin piece for an external catheter formed in accordance with an embodiment.

FIG. 2 is a side view of the three versions of a groin piece for an external catheter shown in FIG. 1.

FIG. 3 is a side cross-sectional view of the three versions of a groin piece for an external catheter shown in FIG. 1.

FIG. 4 is a side and a front view of the first version of a groin piece for an external catheter shown in FIG. 1.

FIG. 5 is a front perspective view of the first version of the groin piece for an external catheter shown in FIG. 4 and having a locking ring configured to secure thereto.

FIG. 6 is a side and a front view of the second version of a groin piece for an external catheter shown in FIG. 1.

FIG. 7 is a side and a front view of the third version of a groin piece for an external catheter shown in FIG. 1.

FIG. 8 is a front perspective view of a fourth version of a groin piece for an external catheter formed in accordance with an embodiment.

FIG. 9 is a side view of the groin piece for an external catheter shown in FIG. 8.

FIG. 10 is a top and a side view of a disposable tube for an external catheter.

FIG. 11 is a top and a side view of a reusable tube for an external catheter.

FIG. 12 is an exploded view of an external catheter formed in accordance with an embodiment.

FIG. 13 is a front perspective view of a strap for an external catheter.

FIG. 14 is a front view of a collection device for an external catheter.

FIG. 15 is a front schematic of an external catheter has a tube extending down the leg of the user.

FIG. 16 is a front schematic of an external catheter having a tube connected to a collection device.

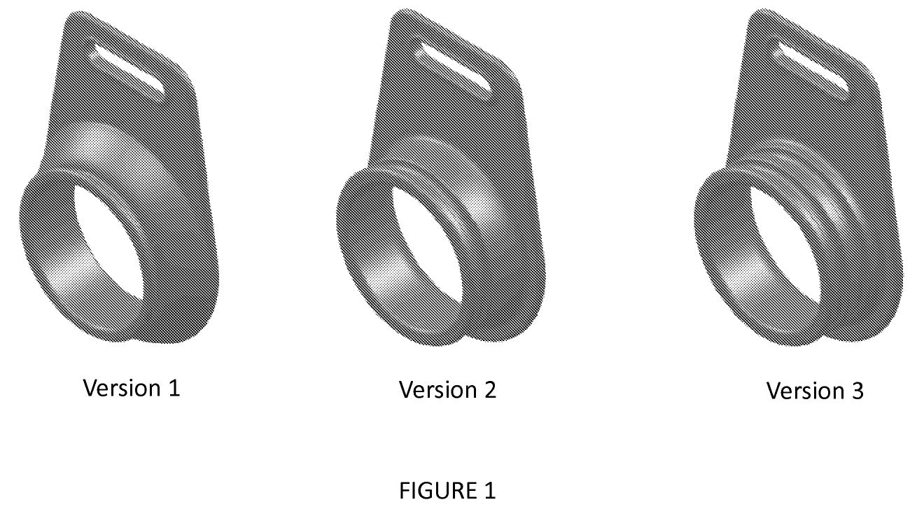

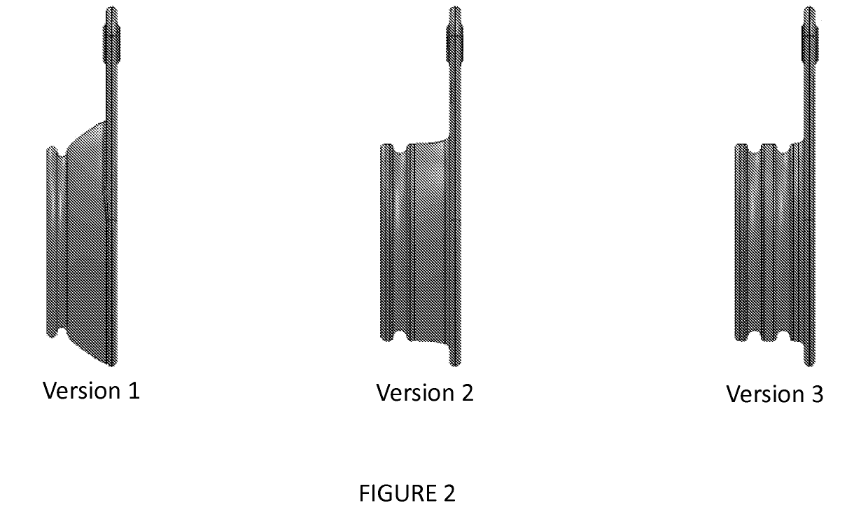

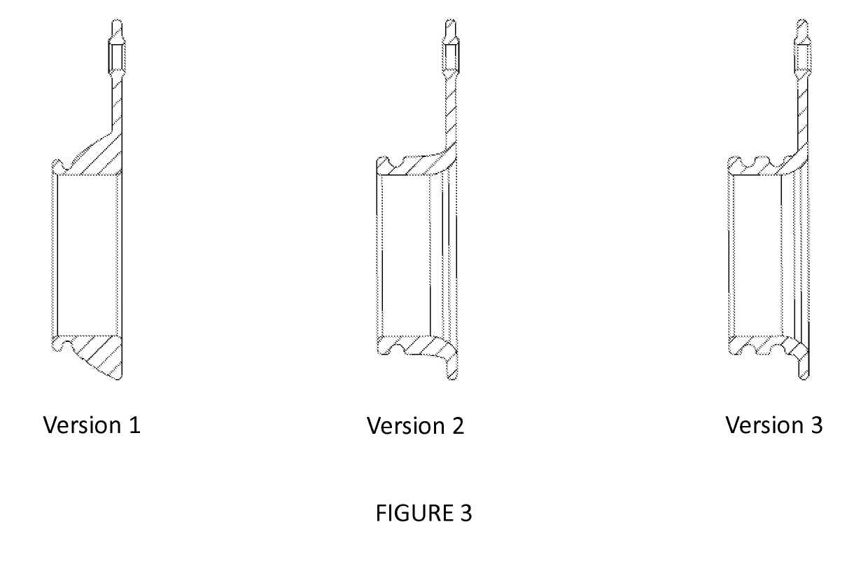

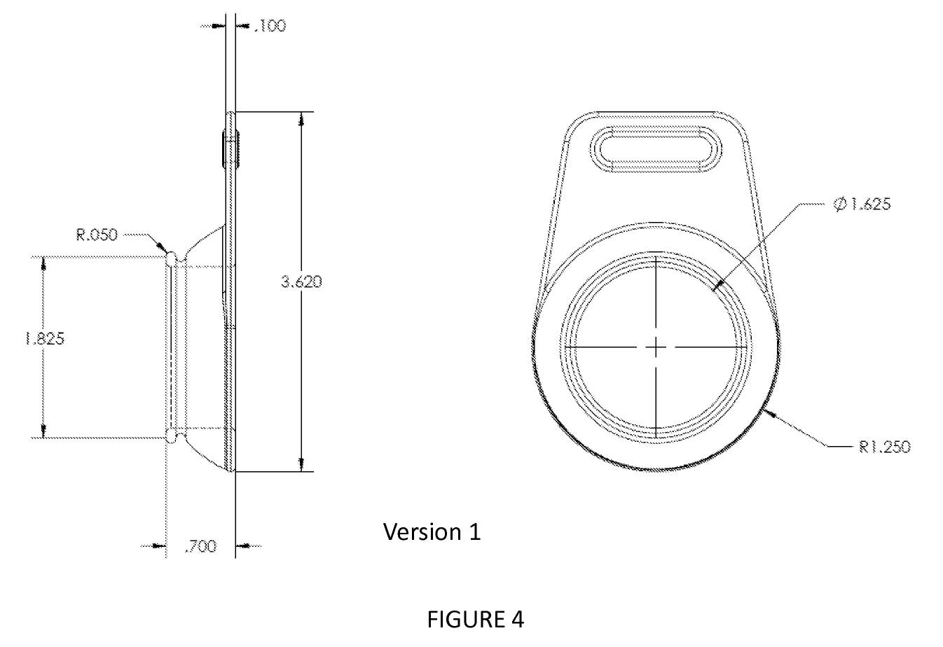

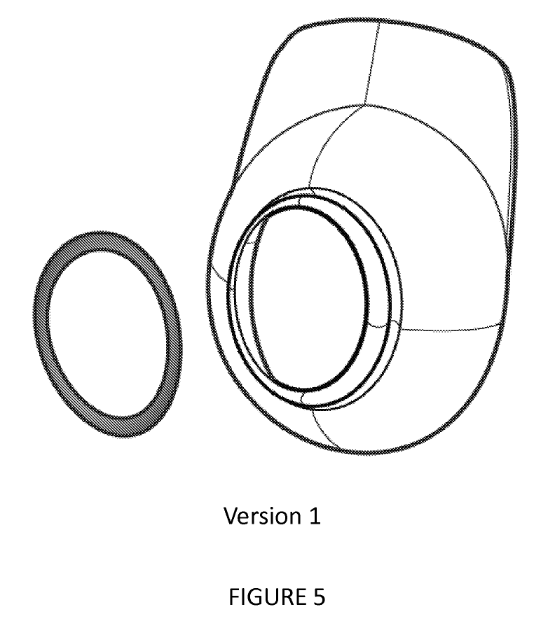

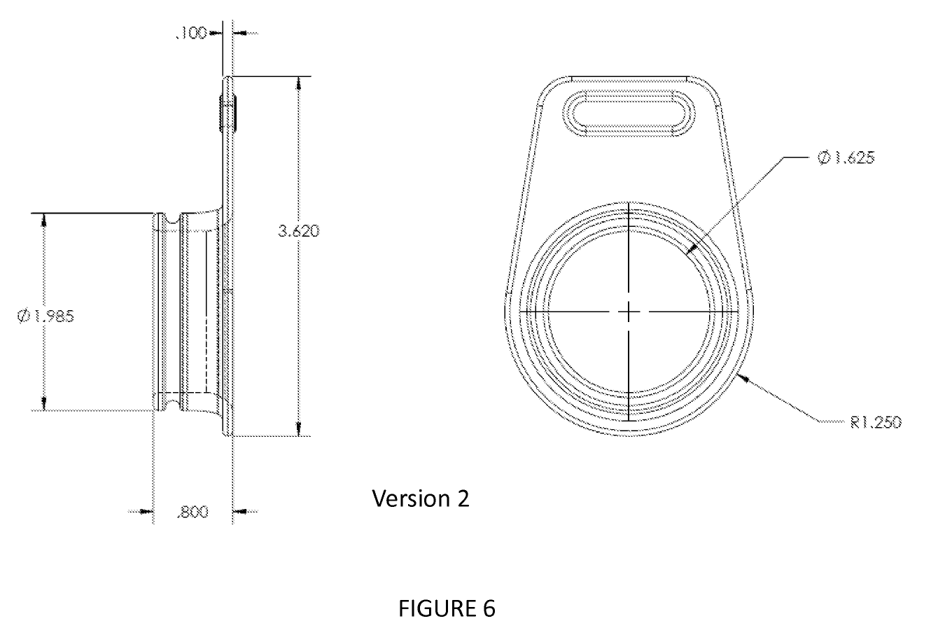

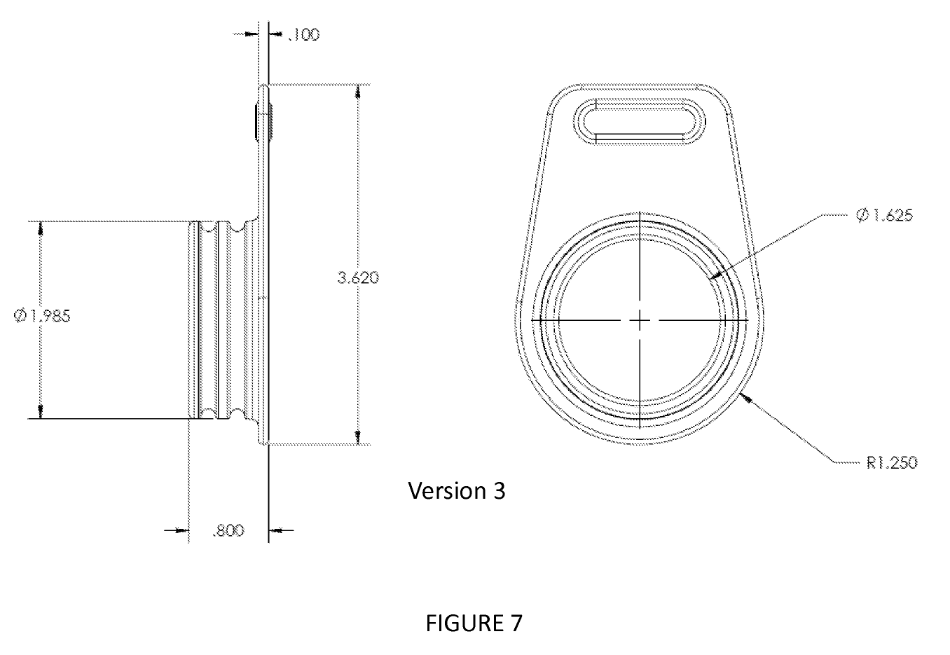

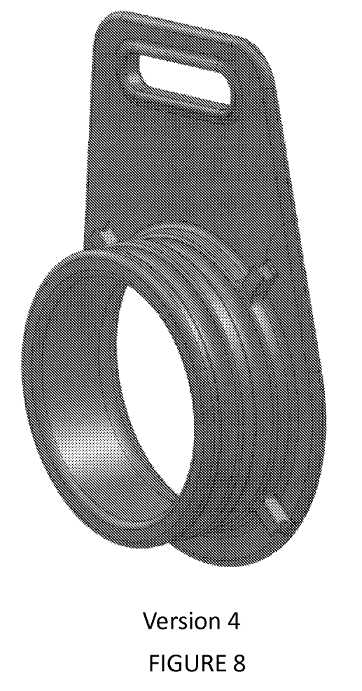

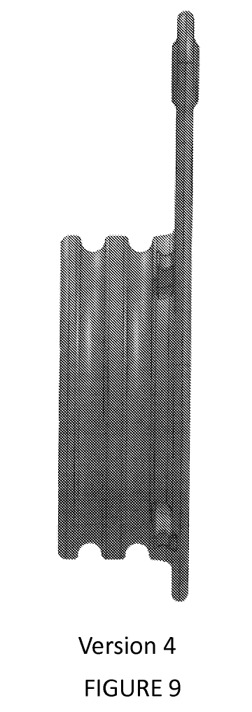

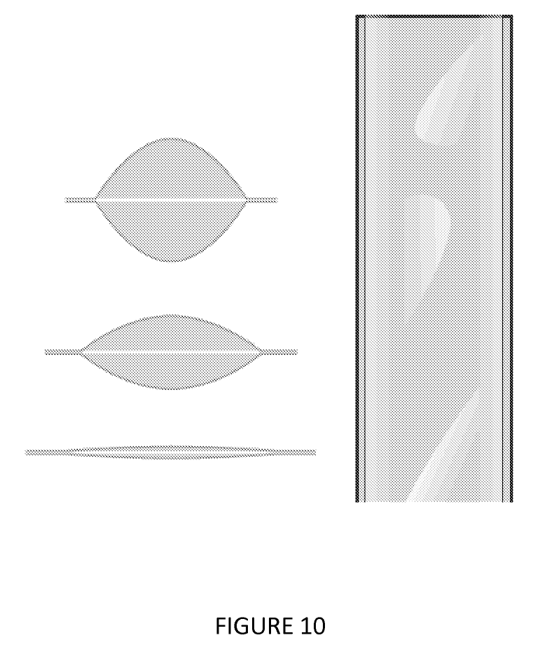

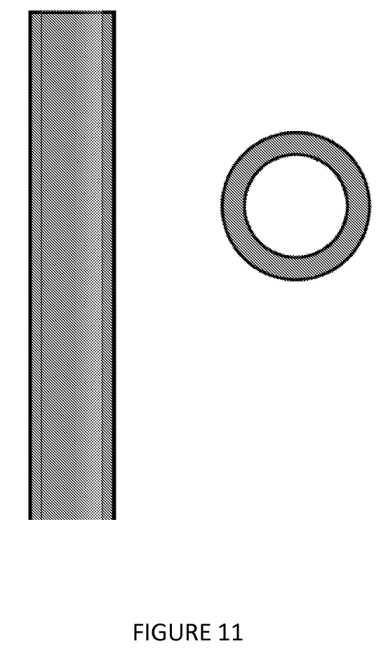

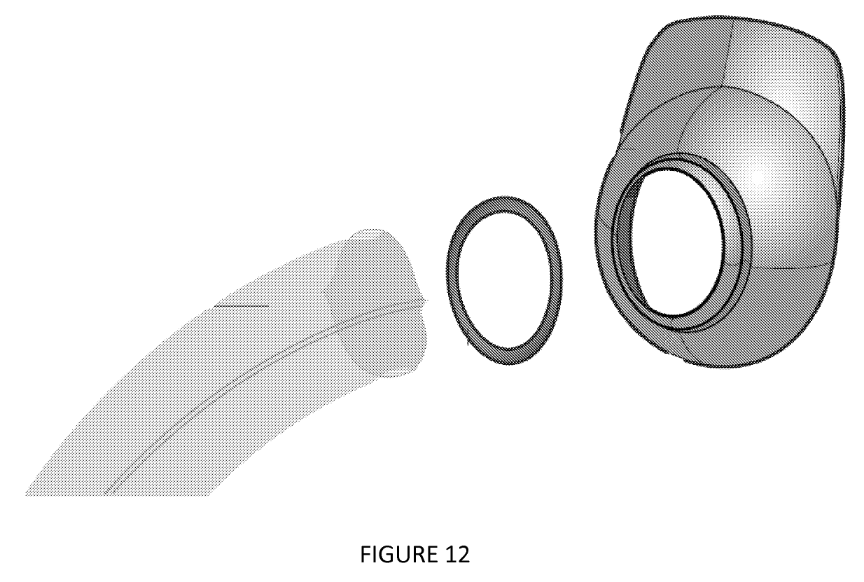

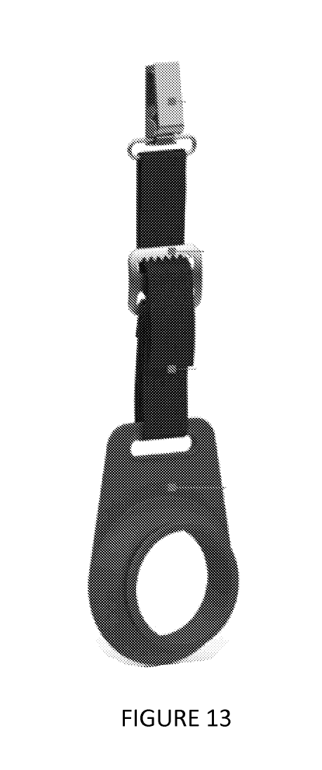

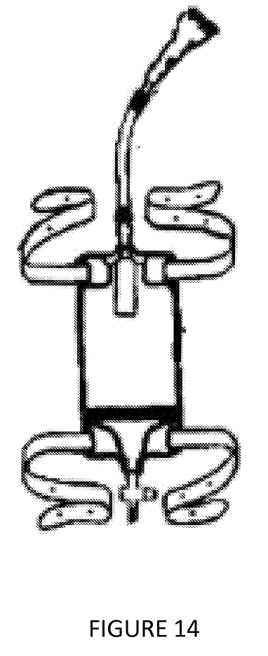

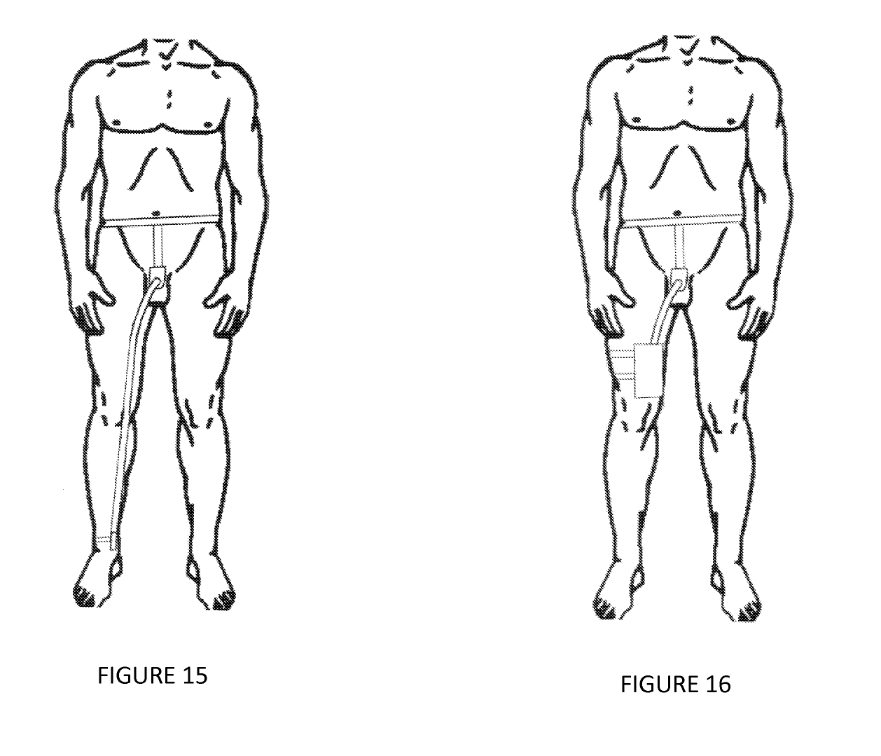

### DETAILED DESCRIPTION OF THE PREFERRED EMBODIMENTS

The following description of the preferred embodiment(s) is merely exemplary in nature and is in no way intended to limit the invention, its application, or uses.

The present invention is an external catheter including a groin piece that is anatomically shaped to a user's pelvis and is sized so that a bottom of the groin piece rests above the user's testicles when the groin piece is positioned on the user's pelvis. The groin piece may include a securing mechanism to secure the external catheter to the user. For example, the securing mechanism may be a strap that is secured the user's belt or a button of the user's pants. A flange extends from the groin piece and defines an opening through the groin piece. The opening is sized to receive the user's penis therethrough. At least one indentation circumvents the flange. A tube is removeably attached to the flange and secured to the flange with a locking ring that is secured in the at least one indentation. Staging pegs may be provided to retain the locking ring when not in use. The tube is preferably disposable and is sized to receive the penis extending through the opening so that a tip of the penis is positioned within the tube distal from the groin piece. The tube has a proximal end and a distal end. In one embodiment, the proximal end is attached to the flange and the tube extends along a length of the user's leg so that the distal end is secured to the user's ankle. In another embodiment, the proximal end is attached to the flange and the distal end is attached to a collection device secured to the user's leg.

The groin piece is preferably made from flexible but rigidly shaped silicone, but may be made from other materials, such as plastic. It is ideal that the groin piece be formed from a material that is easily cleaned and sanitized. The groin piece is anatomically shaped to rest on the user's pelvis without creating a bulge in the user's pants. In particular, the groin piece has a low profile that is generally unnoticed when worn inside of the user's pants. In one embodiment, the groin piece has a thickness of 0.05 to 0.15 inches, and preferably has a thickness of 0.1 inches. Accordingly, the groin piece rests flush on the user's pelvis and generally does not protrude from the user's body. The groin piece may have a length from a top to bottom of 3 inches to 4 inches, and preferably 3.62 inches. The bottom of the groin piece is rounded with a radius of between 1.2 and 1.3 inches, and preferably 1.25 inches. As such, when worn by the user, the bottom of the groin piece rests above the user's testicles. The groin piece is sized to position within any type of undergarments without stretching the undergarment or making the undergarment uncomfortable.

The top of the groin piece may be joined to a securing mechanism. In the illustrated embodiment, the top of the groin piece includes a slot extending therethrough to receive a strap. One end of the strap is secured to the groin piece through the slot. An opposite end of the strap includes an attachment mechanism to secure the groin piece to an article of the user's clothing. For example, the opposite end of the strap may include a loop or clamp that secures to the user's belt. Alternatively, the opposite end of the strap may include a button hole to secure the strap to a button on the user's pants. The strap in generally sized to be pulled through the zipper opening of the users pants and secured to a belt or button. The strap may also be adjustable to accommodate men of various sizes. The strap may be shortened or lengthened to properly position the groin piece at the user's penis.

In another embodiment, the groin piece may include several straps in a jock strap configuration. The groin piece itself is hung from a waistband extending around the user's waist. A pair of straps extends from a bottom of the groin piece and each strap extends around one of the user's legs. The straps are joined to a backside of the waistband to create a wearable garment having the groin piece integrally formed therein. Optionally, the groin piece may be sewn into a standard pair of undergarments or boxer briefs.

A circular flange is formed proximate to the bottom of the groin piece. The flange defines an opening through the groin piece. The opening is sized to receive the user's penis therethrough. In one embodiment, the opening has an inner diameter of 1.5 to 1.7 inches, and preferably 1.625 inches. The penis is extended entirely through the opening so that the groin piece is positioned at the base of the penis and the tip of the penis is positioned distally from the groin piece.

The present invention includes at least four embodiments of the groin piece. In a first embodiment (Version 1), the flange includes a base portion that tapers inward from the groin piece to a locking portion of the flange. The locking portion of the flange extends outward from the base portion so that the entire flanges extends 0.6 to 0.8 inches, and preferably 0.7 inches, from the user's body. The locking portion includes a rounded lip at the distal end of the flange. The rounded lip has an outside diameter of 1.8 to 1.85 inches, and preferably 1.825 inches. The lip is rounded with a radius of 0.04 to 0.06 inches, and preferably 0.05 inches.

An indentation, which is formed as an annular groove, is formed in the locking portion of the flange adjacent to and between the lip and the base portion of the flange. The indentation circumvents the entire flange and is configured to receive a locking ring therein, as discussed in more detail below. In particular, the locking ring is rolled over the lip and positioned within the indentation. The lip secures the locking ring within the indentation; however, the locking ring may be rolled out of the indentation using force.

A second embodiment (Version 2) of the groin piece includes a flange having a base portion extending outward form the groin piece and a locking portion extending from the base portion, so that the entire flange extends 0.7 to 0.9 inches, and preferably 0.8 inches, from the user's body. The locking portion includes a rounded lip at the distal end of the flange. The rounded lip has an outside diameter of 1.9 to 2.25 inches, and preferably 1.985 inches.

An indentation, which is formed as an annular groove, is formed in the locking portion of the flange adjacent to and between the lip and the base portion of the flange. The indentation circumvents the entire flange and is configured to receive a locking ring therein, as discussed in more detail below. In particular, the locking ring is rolled over the lip and positioned within the indentation. The lip secures the locking ring within the indentation; however, the locking ring may be rolled out of the indentation using force.

A third embodiment (Version 3) of the groin piece includes a flange extending outward from the groin piece and having a rounded lip at its distal end. The rounded lip has an outside diameter of 1.9 to 2.25 inches, and preferably 1.985 inches. The entire flange extends 0.7 to 0.9 inches, and preferably 0.8 inches, from the user's body. Two indentations, formed as annular grooves, are formed in the flange. A first indentation is formed proximate to the lip, and a second indentation is formed proximate to the groin piece. A second lip is formed between the indentations. Each indentation circumvents the entire flange and is configured to receive a locking ring therein, as discussed in more detail below. In particular, the locking ring is rolled over the lips and positioned within one of the indentations. The lips secure the locking ring within the indentation; however, the locking ring may be rolled out of the indentation using force. In one embodiment, a locking ring may be positioned in each indentation.

A fourth embodiment (Version 4) of the groin piece has the same configuration as the third version, but also includes staging pegs to retain the locking ring. The staging pegs extend from the flange between the second indentation and the groin piece. Each staging peg includes an indentation formed therein. When the locking ring is not in use, i.e. when there is no tube attached to the flange, as described below, the locking ring may be positioned in the staging pegs. By positioning the locking ring in the staging pegs a tube may be positioned on the flange without having to first position the locking ring on the tube. Accordingly, once the tube is in place on the flange, the locking ring is rolled off the staging pegs and positioned within one of the indentations to secure the tube to the flange.

The tube is secure to the flange on the outside of the groin piece. The tube is not inserted into the opening and no part of the tube is positioned between the groin piece and the user. The tube is slid onto the flange, and is preferably slid along the flange until a proximal end of the tube is positioned adjacent to the base of the groin piece. The locking ring is then rolled into one of the indentations to secure the tube to the flange. The tube is a foldable thin-walled tube that has pliability. Accordingly, the locking ring deforms a portion of the pliable tube to conform to the shape of and rest along the walls of the indentation, so that the locking ring provides a leak-proof seal between the flange and tube. When using one of versions 1-3, the locking ring is first positioned on the tube and then slid toward the base of the groin piece after the tube is attached to the flange. With respect to version 4, the locking ring is held on the staging pegs while the tube is attached to the flange. After the tube is attached to the flange, the locking ring is rolled off of the staging pegs away from the base of the groin piece and into one of the indentations.

The tube is sized for the average male penis, which has an approximately 1.54 inch diameter. Accordingly, the tube is sized with an approximately 1.9 inch interior diameter. The tube is configured to slide onto the user's penis when the external catheter is in use. In particular, the user extends his penis through the opening in the groin piece and into the tube, which is secured to the groin piece. The tube fits closely around the user's penis so that little to no bulge is formed in the user's pants. The tip of the user's penis is positioned within the tube distally from the groin piece. When the user urinates, the urine only travels through the tube. Accordingly, unlike known external catheters, there is no urine within the groin piece. As such, bacteria and the like are prevented from forming in the groin piece. While it is recommended that the groin piece be washed on a regular basis, the washing is relatively simple because the groin piece is not coming in contact with urine. In one embodiment, the groin piece may be suitable for washing in the dishwasher.

In one embodiment, the tube is formed from a low-density polyethylene (LDPE); however, the tube may be formed from other suitable materials. It is preferred that the tube is formed from a hypo-allergenic material. The tube is disposable and may be discarded after use. Accordingly, there is no need to clean the tube, as is required by other external catheters. Replacement tubes may be sold with the external catheter or separately. The tube is formed as lay-flat tubing that is easily rolled up. Extra tubes may be carried in the user's pocket, backpack, or the like. To use the tube, the tube is expanded from the lay-flat configuration into a cylinder and positioned on the flange of the groin piece, as described above. In another embodiment, the tube may be made from rubber and be reusable.

The tube is configured to extend down the leg of the user. The tubes will generally be provided in 42 inch lengths, which is an appropriate length for most males. Shorter users may cut the tube to an appropriate length. Additionally, longer tubes may be available for taller individuals. The tube is extended through the user's pant leg to the user's foot. Straps may be provided to secure the tube along the length of the user's leg. A distal end of the tube is secured, preferably by a strap, at an end of the user's leg at the ankle or foot. In one embodiment, a hard plastic end piece, in the shape of a tube, may be joined to the distal end of the tube. The plastic end piece may include the straps used to secure the tube to the user's leg. The plastic end piece may be removable from the distal end of the tube and may be reusable. The plastic end piece may also be washable by hand or in the dishwasher. By securing the tube, the tube does not shift position as the user walks or sits down.

In one embodiment, the external catheter may be provided with a collection device that is secured to the user's leg. As such, the external catheter is suitable for individuals who are incontinent or have other medical needs for a urine collection device. The collection device may include a valve to empty the urine therein into a toilet or the like.

The embodiments were chosen and described to best explain the principles of the invention and its practical application to persons who are skilled in the art. As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1. An external catheter comprising:
a groin piece that is anatomically shaped to a user's pelvis;
a flange extending from the groin piece and defining an opening through the groin piece, the opening being sized to receive the user's penis therethrough; and
a tube removeably attached to the flange, the tube being sized to receive the penis extending through the opening.

2. The external catheter of claim 1 further comprising a locking ring to secure the tube to the flange.

3. The external catheter of claim 2, wherein the flange includes at least one indentation to receive the locking ring to secure the tube to the flange.

4. The external catheter of claim 2 further comprising staging pegs extending from the flange, the staging pegs having an indentation to retain the locking ring when the ring is not in use.

5. The external catheter of claim 1, wherein the tube is disposable.

6. The external catheter of claim 1, wherein the tube has a proximal end and a distal end, the proximal end attached to the flange, the tube extending along a length of the user's leg, the distal end being secured to the user's ankle.

7. The external catheter of claim 1, wherein the tube has a proximal end and a distal end, the proximal end attached to the flange and the distal end attached to a collection device secured to the user's leg.

8. The external catheter of claim 1 further comprising a securing mechanism attached to the groin piece to secure the external catheter to the user.

9. The external catheter of claim 8, wherein the securing mechanism includes a strap that is secured to at least one of the user's belt or a button of the user's pants.

10. The external catheter of claim 1, wherein the groin piece is sized so that a bottom of the groin piece rests above the user's testicles when the user's penis is extended through the opening.

11. An external catheter comprising:
a groin piece that rests on a user's pelvis;
a flange extending from the groin piece and defining an opening through the groin piece, the opening being sized to receive the user's penis therethrough; and
a disposable tube removeably attached to the flange, the tube being sized to receive the penis extending through the opening so that a tip of the penis is positioned within the tube distal from the groin piece.

12. The external catheter of claim 11 further comprising:
at least one indentation formed around the flange; and
a locking ring positionable in the at least one indentation to secure the tube to the flange.

13. The external catheter of claim 11, wherein the tube has a proximal end and a distal end, the proximal end attached to the flange, the tube extending along a length of the user's leg, the distal end being secured to the user's ankle.

14. The external catheter of claim 11, wherein the tube has a proximal end and a distal end, the proximal end attached to the flange and the distal end attached to a collection device secured to the user's leg.

15. The external catheter of claim 11 further comprising a strap attached to the groin piece to secure the external catheter to the user, the strap being securable to at least of the user's belt or a button of the user's pants.

16. The external catheter of claim 11, wherein the groin piece is sized so that a bottom of the groin piece rests above the user's testicles when the user's penis is extended through the opening.

17. An external catheter comprising:
a groin piece that is anatomically shaped to a user's pelvis and rests above the user's testicles;
a flange extending from the groin piece and defining an opening through the groin piece, the opening being sized to receive the user's penis therethrough;
at least one indentation formed around the flange;
a removable tube attached to the flange, the tube being sized to receive the penis extending through the opening; and
a locking ring positionable in the at least one indentation to secure the tube to the flange.

18. The external catheter of claim 17, wherein the tube is disposable.

19. The external catheter of claim 17, wherein the tube has a proximal end and a distal end, the proximal end attached to the flange, the tube extending along a length of the user's leg, the distal end being secured to the user's ankle.

20. The external catheter of claim 17, wherein the tube has a proximal end and a distal end, the proximal end attached to the flange and the distal end attached to a collection device secured to the user's leg.
